(19) United States
(12) Patent Application Publication
(10) Pub. No.: US 2023/0029178 A1
(43) Pub. Date: Jan. 26, 2023

(54) DRESSING COMPRISING WATER RESISTANT VISUAL INDICIA

(71) Applicant: 3M INNOVATIVE PROPERTIES COMPANY, St. Paul, MN (US)

(72) Inventor: Caroline J. Hale, Epping North (AU)

(21) Appl. No.: 17/782,487

(22) PCT Filed: Dec. 7, 2020
(86) PCT No.: PCT/IB2020/061589

§ 371 Date: Jun. 3, 2022

(30) Foreign Application Priority Data
Dec. 13, 2019 (AU) 2019280098

(51) Int. Cl. A61L 15/52; A61F 13/00

(52) U.S. Cl. CPC A61L 15/52; A61F 13/00059; A61F 2013/00319; A61F 2013/00859

(57) ABSTRACT

A wound dressing is disclosed that includes a water resistant visual indicia provided on an area of the dressing. The water resistant visual indicia may be provided on a tag attached to the dressing. The tag may comprise a top sheet and a bottom sheet, the bottom sheet comprising a writeable surface on which the visual indicia is handwritten, or printed.

### TECHNICAL FIELD

The present disclosure relates to medical dressings comprising water resistant visual indicia.

### BACKGROUND

Wound dressings used by civilian or hospital emergency response teams are typically deployed at the site of accidents or emergencies, in which the environment may include dirt, grime, sweat and other elements that can stay trapped in the dressing and cause an infection in the wound. In instances where wound dressings are applied in a sub-optimal or a non-aseptic environment, hospitals usually require that they be replaced promptly. By comparison, wound dressings applied in a hospital are in a cleaner environment and only needs to be changed when soiling occurs. However, wound dressings used in both situations may be identical, making it difficult to visually distinguish between dressings that require replacement and those that do not.

In order to provide a means of identification for wound dressings, personnel may improvise stickers or writings on the dressing to denote dressing conditions. For example, medics may be given colored-coded stickers or blank labels on which to write notes to help them identify the dressing as a candidate for replacement within a specified time-frame. While feasible, ad-hoc handwritten markings on dressings may smudge in the rain or sweat or other bodily fluids, thereby becoming illegible. Furthermore, adhesive labels deployed in a wet environment may not stick well in a wet environment. This increases the burden of the medical personnel tasked to carry out this additional step amidst a potentially chaotic environment and alongside other dressing application steps.

Therefore, there is a need for a more systematic and convenient way of marking dressings.

### SUMMARY

In one aspect, the present disclosure provides a wound dressing comprising a water-resistant visual indicia provided on a surface of the dressing.

In another aspect, the present disclosure provides a method of identifying a patient, comprising reading the water-resistant visual indicia on a wound dressing. In an embodiment, the visual indicia indicates the application environment of the wound dressing.

The techniques described herein shall be understood by the skilled person to be capable of variations and modification without departing from the essence of the claimed invention. Accordingly, the drawings and description as follows shall be regarded as illustrative.

### BRIEF DESCRIPTION OF DRAWINGS

Reference will now be made to exemplary embodiments as illustrated with reference to the accompanying figures. The figures together with a detailed description below, are incorporated in and form part of the specification, and serve to further illustrate the embodiments and explain various principles and advantages, in accordance with the present disclosure.

FIG. 1 is a top view of a wound dressing comprising visual indicia.

FIG. 2 is a top view and a cross sectional view of a wound dressing comprising visual indicia protected by a tag.

FIG. 3 is a perspective view and a cross-sectional of a wound dressing comprising a label inserted in a tag.

FIG. 4 is a top view of an intravenous (IV) catheter securement dressing comprising visual indicia.

FIG. 5 is a perspective view showing the layer structure of the IV catheter securement dressing comprising visual indicia.

FIG. 6 shows dressings bearing textual and machine-readable code visual indicia.

FIG. 7 is a perspective view of an IV catheter securement dressing bearing a plurality of different types of visual indicia.

### DETAILED DESCRIPTION

The present disclosure provides wound dressings comprising water resistant visual indicia arranged on a visible area of the dressing. Such wound dressings have the benefit of being adapted to carry visual information without risk of the external environment damaging the information being carried. Visual indicia may comprise machine readable code, enabling healthcare providers to attach parameters such as date, time, product code, time elapsed since dressing application, patient information, and so on, directly onto the dressing. By rendering the visual indicia water resistant, the wound dressings become readily deployable in both wet and dry environments, reducing the risk of soiling the visual indicia.

In this document, the following words and terms used herein shall have the following meaning:

The terms “water resistant”, “waterproof” and “water repellant” are used interchangeably and refer to the ability to resist the penetration of moisture or water at least to some degree. It also encompasses the ability to remain unaffected by the presence of water not withstanding direct contact with water.

The word “substantially” does not exclude “completely” e.g. a composition which is “substantially free” from a substance may be completely i.e. 100%, free from the substance.

Unless otherwise specified, the terms “comprising” and “comprise”, are intended to denote inclusivity such that a device comprising a set of recited element would not only include the recited elements, but also includes other additional, unrecited elements.

As used herein, the term “about”, in the context of physical dimensions, or chemical concentrations of components of the formulations, depending on the experience and skill of the technician, means up to +/−10% of the stated value, and more typically should vary between +/−5%, +/−4%, +/−3%, +/−2% or +/−1% of the stated value.

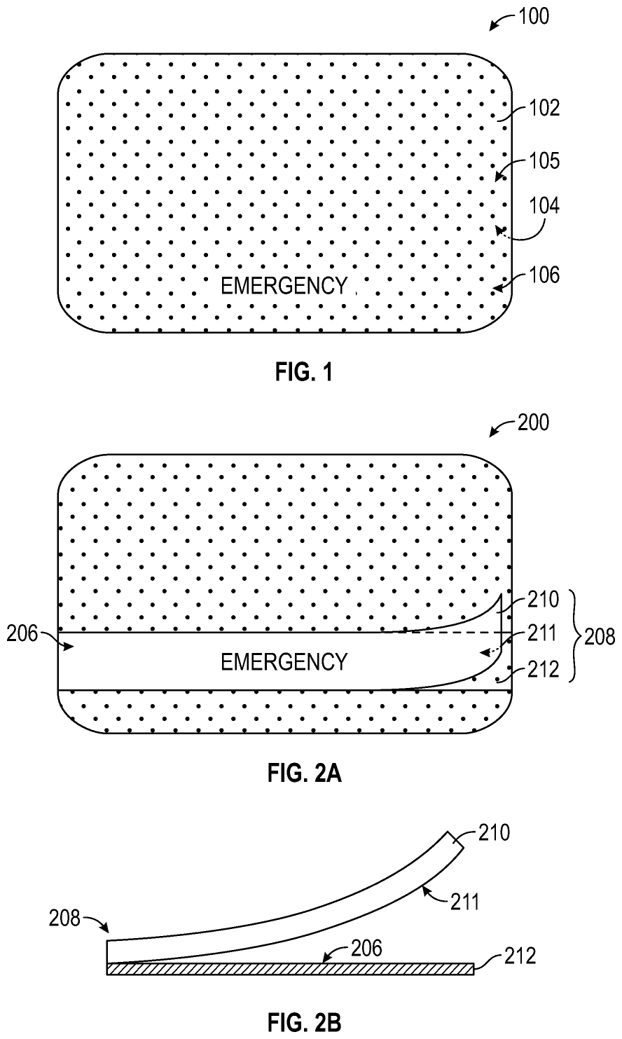

Referring to FIG. 1, in an embodiment, wound dressing 100 comprises a sheet assembly 102 comprising, a sheet of suitable dressing material 104 supported by a substrate 105. The dressing material is intended to be on the skin-facing side and may comprise hydrogel, linen or non-woven material. The substrate provides mechanical support to the dressing material, and may be made of any suitable flexible material, which may be water proof or water permeable, such as a polymer film, which may be made from polymers such as polyvinylchloride (PVC), polyethylene (PE), polypropylene (PP) or polyurethane (PU), or a natural latex sheet. For improved breathability, the substrate may be provided with openings. The skin-facing side of the dressing and substrate may comprise pressure sensitive adhesive for attaching the wound dressing to the skin.

In accordance with the present disclosure, visual indicia 106 is provided on a visible area of the dressing. In this embodiment, textual information is handwritten (or printed) on the top surface of the dressing. The top surface of the dressing may be adapted for water resistance by coating the substrate surface with a water resistant coating. In some embodiments, a hydrophobic coating or low surface energy coating may be applied. For example, an alcoholic suspension of SiO₂ nanoparticles may be sprayed onto the area after the visual indicia has been printed or written. Fluorinated silanes and fluoropolymer coatings may also be used. A commercially available example of a usable hydrophobic coating includes 3M™ Scotchgard™ Heavy Duty Water Shield.

In this example, the visual indicia comprises the hand-written word “EMERGENCY”, which may serve to indicate to healthcare personnel of the fact that the dressing was administered to the patient in an emergency situation and can help to remind them that the dressing needs to be inspected regularly and changed, if necessary. More generally, the visual indicia may relate to a condition of the environment under which the dressing was applied onto a subject. In an embodiment, the visual indicia relates to patient information or an address or link to such patient information. Any other textual information may be used in place of the word “EMERGENCY” as may be appropriate. For water resistance, inks compatible with the water resistant surface of the dressing should be used. Marker pens which dispense waterproof pigment inks in organic solvents (e.g. toluene, xylene or alcohol) may be used to write such visual indicia.

In a preferred embodiment, the visual indicia is machine-printed onto the top surface of the dressing. In order to render the visual indicia water resistant, a water-insoluble printing ink may be used to print the visual indicia in conjunction with an inkjet printer or screen printing methods. Inks usable for this purpose include aqueous water insoluble inorganic inks, as well as inks comprising organic pigments and resins dissolved in an organic solvent. Commercially available inks include 3M™ Screen Printing Ink Series 1900, or Series 2900 inks, as well as 3M™ Reflective Ink for Textiles—8010. The visual indicia may also be laser printed using carbon black toner.

To enable the visual indicia on the dressing to withstand rain, perspiration, as well as to protect it from patient-tampering, dirt and abrasion, it is preferably provided on a water impermeable tag attached to a surface of the dressing. In am embodiment, the tag comprises a top sheet and a bottom sheet, the bottom sheet comprising a writeable surface on which the visual indicia is handwritten and the top sheet comprising a self-adhesive transparent film. comprising a writeable surface for receiving a water-resistant ink. In another embodiment, the visual indicia is written onto the tag and then sealed with a cover sheet. Referring to FIG. 2, a top view of a dressing 200 is shown comprising visual indicia 206 sealed within a tag 208. Bottom sheet 212 serves as a writeable area on which visual indicia 206 may be written or printed. Bottom sheet 212 is preferably a water-resistant material so that exudate from an underlying wound does not penetrate the tag from beneath to smudge the visual indicia. The writable layer surface may be treated to render it suitable for receiving a water-based ink, or preferably, to receive organic solvent-based pigment inks. Top sheet 210 is preferably transparent and has a pressure sensitive adhesive layer 211 on its bottom-facing surface initially covered by a release liner (not shown), and after the visual indicia is written or printed, the release liner may be removed and the top sheet 210 is pressed onto the bottom sheet 212 to seal the visual indicia within, rendering it impervious to water. Such a tag may come manufactured with the dressing, or it may be included as part of a kit where self-adhesive tags are to be manually attached to the dressing.

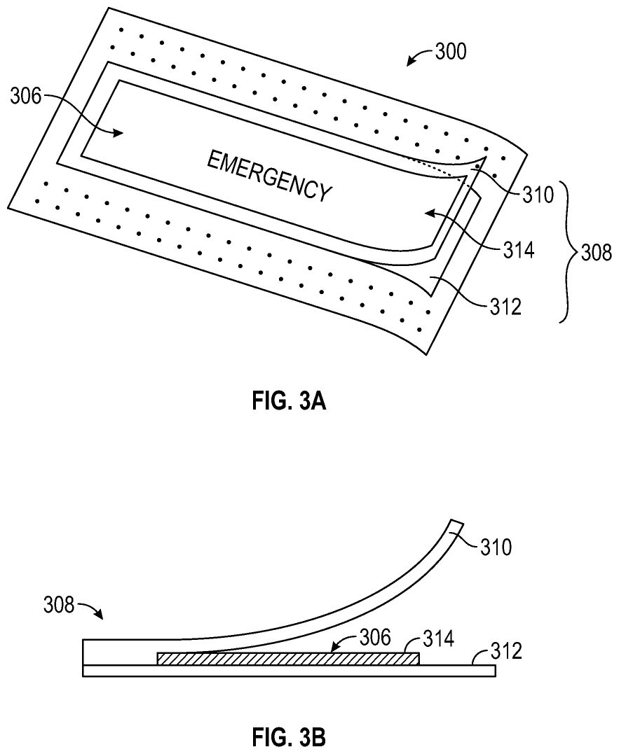

In some situations, it may be desirable to have the visual indicia pre-printed onto a sheet label and then inserted within the tag. This allows the visual indicia to be mass printed beforehand and distributed to personnel for use with sealable tags on a particular incident or date, for example. FIG. 3 shows the top view and cross-sectional view of a possible construction that facilitates the use of such printed labels. Tag 308 comprises visual indicia 306 written on a label 314. The tag 308 comprises a top sheet 310 and bottom sheet 312. The label 314 to be used is inserted between the top sheet 310 and bottom sheet 312. Pressure sensitive adhesive is provided on the bottom-facing surface of top sheet 310. When the visual indicia is ready to be sealed, the release liner covering the adhesive is removed and the top sheet 310 is pressed onto the bottom sheet 312, sealing the visual indicia.

The present disclosure is applicable to any sort of wound dressing requiring labeling, and is particularly advantageous for deployment in the field, during situations of emergencies, accidents and disasters, either during wet or normal weather. Examples include bandages and Carlisle type first aid dressings (FAD), as well as gauze, films, foams and composite dressings applied in the hospitals.

In an embodiment, the wound dressing comprises intravenous (IV) catheter securement dressings. An IV catheter is a hollow needle device used to provide fluids such as blood, medication, or other fluids to a patient. Following insertion of the catheter into a patient, e.g. patient's vein via the skin, it is secured to the patient's skin with a dressing. A securement dressing is normally applied to the catheter insertion site to secure the catheter to the patient to prevent unintended removal of the catheter during the patient's movement. It also helps to minimize discomfort and to allow for subsequent connection to a fluid reservoir as needed. Furthermore, the securement dressing limits exposure of the catheter site to external elements, thereby preventing infection of the puncture wound.

IV catheters are frequently employed in both field situations and in the hospitals for the purpose of intravenously supplying fluids such as saline, pharmaceutical drugs, and water to the injured or sick via a catheter inserted into the arm. Conditions in the field often involve rain, dirt and grime, and visual indicia may be provided denoting the dressing as requiring change. In an IV catheter dressing, the dressing may comprise a transparent section comprising a flexible substrate layer, and an opaque section comprising a dressing layer, the dressing layer being attached to a portion of the flexible layer and configured with an incision from an edge thereof, the sheet assembly having a top-oriented surface and an opposing bottom-oriented surface, an adhesive disposed on the bottom surface of the sheet assembly, and at least one visual indicia is printed and arranged over the opaque section of the sheet assembly, wherein the visual indicia indicates the application environment of the wound dressing.

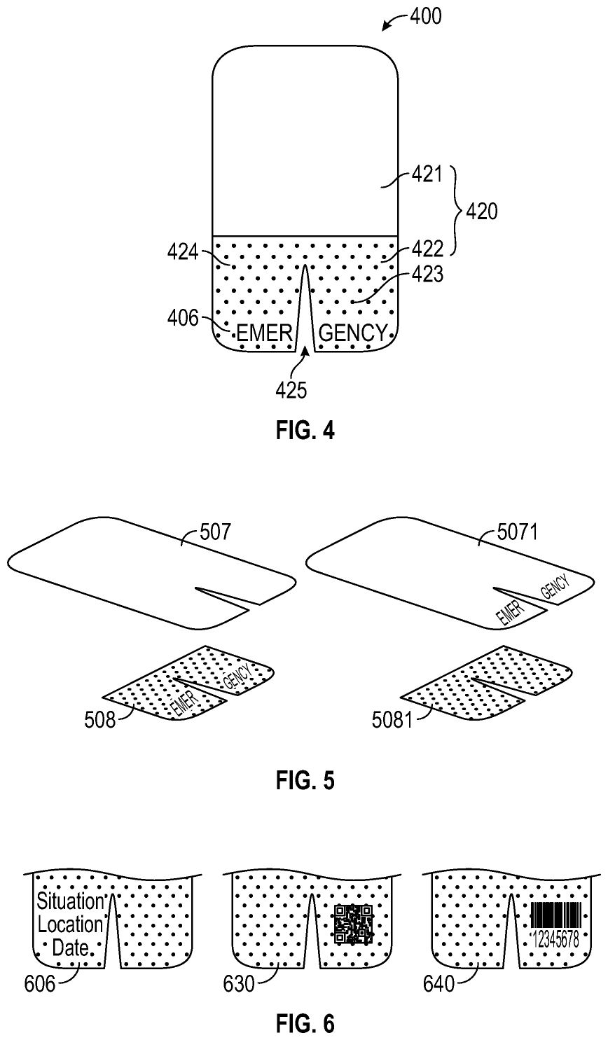

FIG. 4 shows an IV catheter securement dressing in accordance with an embodiment. Catheter securement dressing 400 comprises a sheet assembly 420 having a transparent section 421. The transparent section may comprise a transparent flexible polymer film, such as polyurethane, polyethylene or polypropylene. Adjacent to the transparent section is an opaque section 422, which comprises a dressing material layer. The dressing layer may comprise non-woven fabric such as non-allergenic polyester. Transparent section 421 is intended to be placed over the catheter puncture site on a patient after insertion of the catheter. Its transparency provides visual access of the condition of the catheter and the puncture wound on the patient's skin. The dressing layer is configured with an incision 425 starting from an edge of the dressing layer and extending to a terminal position near the transparent section 421. This incision 425 is intended to provide a recess for the placement of the catheter. The left and right flaps 423, 424 of the dressing layer is intended to provide a padded support surface for structural features of the catheter which may rest on the patient's skin, and may thus help to reduce any unwanted abrasion or pressure on the patient's skin.

Visual indicia 406 may be printed or handwritten onto an IV securement dressing in accordance with any of the embodiments described above. In greater detail, as shown in FIG. 5, the sheet assembly 500 may comprise a top layer 507 and an opposing bottom layer 508 on which the visual indicia is printed. Alternatively, in another embodiment, the visual indicia is printed and arranged over the top layer 5071 instead of the bottom layer 5081.

Other informational aspects of the dressing, the environment relating to the application of the dressing, or the subject's condition, or any other information, may be specified in place of the word “EMERGENCY”. Examples include words denoting the situation, location or date, and such visual indicia 606 may be printed on the securement dressing, or any other suitable location on the dressing, as shown in FIG. 2A. The visual indicia may be printed in other formats such as a repeating pattern or in smaller or bigger fonts.

In another aspect, the present disclosure provides a method of identifying a patient, comprising reading the visual indicia on a wound dressing as defined in the present disclosure. In a embodiment, the visual indicia indicates the application environment of the wound dressing. In another embodiment, the visual indicia comprises a machine-readable link to patient information stored on a server. In the daily operation of hospitals and clinics, productivity may be improved by equipping hospital personnel with dressings that include visual indicia, such as tags that can be mass-printed daily with the required information and attached to the dressing for use throughout each day. Rapid deployment can be achieved in this manner. Information on the tag may help nurses or medics determine, for example, the date of administration, the identity of the dressing administering personnel, and/or the location of administration. Such information may also include wound care instructions, or circumstances that the victim was in, and so on. The clinic or hospital may determine any other relevant information to be included as visual indicia as part of its standard operating procedure.

In cases where the textual information to be included as visual indicia is too lengthy to be included, it may be desirable to implement symbolic or encoded forms of visual indicia on the dressing instead. Accordingly, in one embodiment, the visual indicia comprises a machine readable code. Examples of a machine readable code includes a 1 dimensional bar code (640 of FIG. 6) of any of the following symbologies: EAN, UPC, GS1 DataBar, Code-128, QR Code, Data Matrix, PDF417, Postal Codes, ISBN. Another example of a machine readable code is a 2 dimensional bar code of any of the following symbologies: Data Matrix, MaxiCode, PDF 417, PDF 417 Truncated, QR-Code (630 of FIG. 6), Swiss QR-Code, Codablock F, and Aztec Code.

In a further embodiment, the visual indicia comprises a barcode for a serial number or an alphabetic code assigned to the conditions surrounding the event, such as the location or date of the event. The barcode may also comprise code representing a stock count of the dressing used, so medical professionals may scan the barcode to check out a piece of dressing from a hospital or medic store, thus automating stock counting for dressings used. In another embodiment, the visual indicia comprises a QR code. The QR code may encode any of the following information: plain text, an email address, an SMS, or a vCard.

In a preferred embodiment, the visual indicia comprises QR code encoding a URL to a landing page of a website or database containing a patient's personal data or healthcare data, such as the patient's personal particulars, or circumstances leading to the patient's injury, or doctors' assessment of the patient's condition, and/or drug administration records, the date of administration of the dressing, the identity of the administering personnel, and/or the location of administration, and so on.

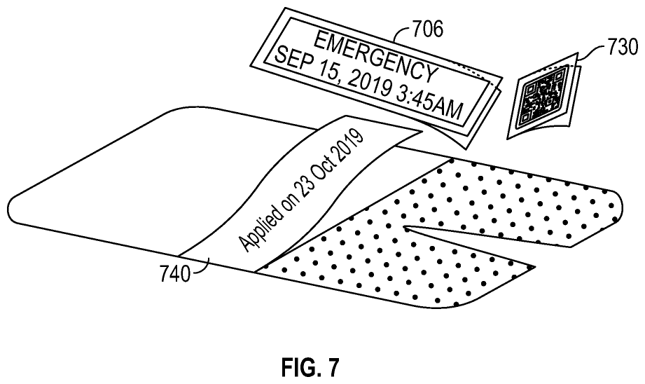

FIG. 7 shows another embodiment in which a plurality of visual indicia is provided on a wound dressing. The plurality of visual indicia may be provided on one or more of a combination of tags, writeable surfaces and directly printed visual indicia, as may be needed.

Various embodiments and implementation of the present disclosure have been disclosed. The disclosed embodiments are presented for purposes of illustration and not limitation. The implementations described above and other implementations are within the scope of the following claims. One skilled in the art will appreciate that the present disclosure can be practiced with embodiments and implementations other than those presented herein. Those having skill in the art will appreciate that many changes may be made to the details of the above-described embodiments and implementations without departing from the underlying principles thereof. The scope of the present application should, therefore, be determined only by the following claims.

1. A wound dressing comprising water-resistant visual indicia provided on a visible area of the dressing.

2. The dressing of claim 1, wherein the visual indicia relates to a condition of the environment under which the dressing was applied onto a subject.

3. The wound dressing of claim 1, wherein the visible area of the dressing on which the visual indicia is provided comprises a hydrophobic coating or low surface energy coating.

4. The wound dressing of claim 1, wherein the visual indicia is comprised in a water impermeable tag attached to a surface of the dressing.

5. The wound dressing of claim 4, wherein the tag comprises a top sheet and a bottom sheet, the bottom sheet comprising a writeable surface on which the visual indicia is handwritten and the top sheet comprising a self-adhesive transparent film.

6. The wound dressing of claim 5, wherein the tag further comprises a printed label carrying a printed visual indicia inserted between the top sheet and the bottom sheet.

7. The dressing of claim 1, wherein the water-resistant visual indicia comprises machine-readable code.

8. The dressing of claim 7, wherein the machine-readable code comprises a QR code.

9. A dressing comprising:
a sheet assembly comprising a transparent section comprising a flexible substrate layer, and an opaque section comprising a dressing layer, wherein the dressing layer is attached to a portion of the flexible substrate layer and configured with an incision from an edge thereof, wherein the sheet assembly has a top-oriented surface and an opposing bottom-oriented surface,
an adhesive disposed on the bottom surface of the sheet assembly, and
at least one visual indicia printed and arranged over the opaque section of the sheet assembly, wherein the visual indicia indicates the application environment of the wound dressing.

10. The dressing of claim 9, wherein the dressing comprises an intravenous IV catheter securement dressing.

11. The dressing of claim 9, wherein the adhesive comprises a pressure sensitive adhesive.

12. The dressing of claim 9, wherein the dressing layer comprises non-woven fabric comprises non-allergenic polyester.

13. A method of identifying a patient, comprising: reading visual indicia on a wound dressing of claim 1.

14. The method of claim 13, wherein the visual indicia indicates the application environment of the wound dressing.

15. The method of claim 13, wherein the visual indicia comprises a machine-readable link to patient information stored on a server.
